(19) United States
(12) Patent Application Publication
(10) Pub. No.: US 2009/0136742 A1
(43) Pub. Date: May 28, 2009

(54) Ag-containing solution, antibacterial resin composition comprising the solution and antibacterial resin coated steel plate

(75) Inventors: Hyun-Tai Kim, Kyungsangbook-do (KR); Jin-Tae Kim, Kyungsangbook-do (KR); Yeong-Sool Jin, Kyungsangbook-do (KR)

Correspondence Address: THE WEBB LAW FIRM, P.C., 700 KOPPERS BUILDING, 436 SEVENTH AVENUE, PITTSBURGH, PA, 15219, US

(73) Assignee: POSCO, Pohang (KR)

(21) Appl. No.: 11/920,902

(22) PCT Filed: May 23, 2006
(86) PCT No.: PCT/KR2006/001932

§ 371 Date: Nov. 21, 2007

(30) Foreign Application Priority Data
May 25, 2005 (KR) 10-2005-0044146

(51) Int. Cl. C09D 7/12; A61K 33/38; B32B 15/08

(52) U.S. Cl. 428/336; 424/618

(57) ABSTRACT

An aqueous silver-containing solution comprising nano-sized silver particles wherein silver (Ag) particles having a particle diameter of 1 to 20 nm have a concentration of 200 to 100,000 ppm, the pH of the solution is maintained in a range of 6 to 8.5, and based on the weight of the silver-containing solution, an amount of a stabilizer as an impurity is in a range of 0.5 to 1.5% by weight, an amount of anions of a silver salt as another impurity is 1.0% by weight or less and the total amount of the stabilizer and anions of the silver salt is 2.0% by weight or less; an antibacterial resin composition comprising 100 parts by weight of an acrylic, urethane, epoxy or ester resin, 0.05 to 5 parts by weight of a curing agent and a silver-containing solution in an amount such that a concentration of silver is in a range of 5 to 100 ppm, relative to the resin composition; and an antibacterial resin-coated steel plate having a dry film thickness of not more than 5 μm which is formed by coating of the aqueous antibacterial resin composition. Steel plates coated with the antibacterial resin composition exhibit superior antibacterial properties, corrosion resistance, conductivity and adhesion.

### TECHNICAL FIELD

The present application is based on, and claims priority from, Korean Application Number 2005-44146, filed May 25, 2005, the disclosure of which is incorporated by reference herein in its entirety.

The present invention relates to a silver-containing solution, an antibacterial resin composition comprising the same and being capable of imparting superior antibacterial properties and anticorrosiveness to a steel plate, and a steel plate coated with a resin composition. More specifically, the present invention relates to an aqueous silver-containing solution comprising nano-sized silver particles, an antibacterial resin composition comprising the same and being capable of imparting superior antibacterial activity, corrosion resistance, conductivity and adhesion to a steel plate, and a steel plate coated with a thin film of such an antibacterial resin composition.

### BACKGROUND ART

Conventionally, in order to secure corrosion resistance of steel plates coated with a thin-film of a resin, steel plates or zinc-galvanized steel plates have been treated with chromium or chromate. However, chromium is an environmentally hazardous substance and is therefore not currently used. As a result, resin coating is usually used to impart corrosion resistance to steel plates.

As is generally known, a myriad of harmful bacteria inhabit peripheral equipment which is commonly used in daily life. In particular, various bacteria live everywhere moisture and oxygen are available and cause a variety of diseases and disorders.

Further, treatment of the peripheral equipment with the resin results in deterioration of weldability due to non-conductivity of the resin. Consequently, in order to prevent poor weldability resulting from the coating of the resin layer, the resin layer is formed to have as thin a thickness as possible. However, where the resin layer is applied in the form of a thin film, corrosion resistance of the steel plate is undesirably decreased.

As an attempt to solve the problems as discussed above, Japanese Patent Laid-open Publication No. Hei 10-34814 discloses a coated steel plate to which a mixture of a resin and a metal was applied. However, due to the use of an acidic antibacterial agent, the above Japanese Patent Application suffers from weak corrosion resistance of the steel plate when the steel plate is not subjected to chromium or stainless treatment, and therefore requires separate chromium treatment to improve corrosion resistance after a plating process, thus adversely affecting the surrounding environment.

Japanese Patent Laid-open Publication No. Hei 8-156175 discloses an antibacterial-coated steel plate, which is prepared by forming a zinc- or zinc alloy-plating layer on a stainless steel plate and applying a thermosetting coating layer containing an antibacterial agent to the resulting plating layer. However, this patent employs a stainless steel plate as a base metal and disadvantageously suffers from occurrence of white rust.

Japanese Patent Laid-open Publication No. 1998-251557 discloses a resin composition for paints, which comprises an antimicrobial agent prepared by performing ion exchange of a metallic or organic antimicrobial substance having antibacterial activity in flame-retardant phosphate. However, this technique involves essential use of the flame-retardant phosphate which in turn is accompanied by decreased conductivity.

Japanese Patent Laid-open Publication No. 2003-192915 discloses a thermoplastic antibacterial resin composition comprising a silver chloride complex salt, but undesirably poses problems associated with securing of corrosion resistance due to the presence of chlorine ions.

Japanese Patent Laid-open Publication No. 2003-171604 discloses an antibacterial photocatalytic coating material which is based on a silicone coating material, but this technique cannot be applied to household electric appliances due to poor processability.

Further, Korean Patent Laid-open Publication No. 1996-10736 discloses an antibacterial resin composition containing antibacterial zeolite which was partially replaced with Ag or Zn powder, but this technique is applied to a thick film-type steel plate having a coating thickness exceeding 20 □.

Korean Patent Registration No. 210287 discloses a zinc-galvanized steel plate wherein an antibacterial resin layer of the steel plate contains silver and an ester resin is used as a base material, but the antibacterial layer is formed of two layers and a chromate-treated steel plate is used as a base steel plate.

In addition, there are numerous products such as PCM steel plates in which antibacterial properties were imparted to zinc-galvanized steel plates or a resin treated pipe or the like. Most of these products are provided with antibacterial properties by formation of a resin-coated layer having a thick thickness and therefore have no problem in expression of desired corrosion resistance properties but unfortunately do not take into consideration conductivity and/or adhesion.

### DISCLOSURE OF INVENTION

### Technical Problem

Therefore, the present invention has been made in view of the above problems, and it is an object of the present invention to provide an aqueous silver-containing solution having antibacterial activity. The silver-containing solution in accordance with the present invention is designed to have a concentration of impurities which is specifically controlled, and it is therefore possible to impart superior antibacterial activity, corrosion resistance, conductivity and adhesion to a steel plate via coating of the steel plate with a composition comprising such a silver-containing solution.

It is another object of the present invention to provide an antibacterial resin composition comprising the above silver-containing solution and therefore imparting superior antibacterial activity, corrosion resistance, conductivity and adhesion to a steel plate via application thereof to a steel plate.

It is yet another object of the present invention to provide a resin-coated steel plate which is coated with a thin film of the above antibacterial resin composition and therefore exhibits superior antibacterial activity, corrosion resistance, conductivity and adhesion.

### Technical Solution

In accordance with an aspect of the present invention, the above and other objects can be accomplished by the provision of an aqueous silver-containing solution comprising nano-sized silver particles wherein silver (Ag) particles having a particle diameter of 1.0 to 20 nm have a concentration of 200 to 100,000 ppm, the pH of the solution is maintained in a range of 6 to 8.5, and based on the weight of the silver-containing solution, a content of a stabilizer as an impurity is in a range of 0.5 to 1.5% by weight, a content of an anionic part of a silver salt as another impurity is 1.0% by weight or less and the sum of the stabilizer and anionic part of the silver salt is 2.0% by weight or less.

In accordance with another aspect of the present invention, there is provided an aqueous antibacterial resin composition comprising 100 parts by weight of at least one resin selected from the group consisting of acrylic, urethane, epoxy and ester resins, 0.05 to 5 parts by weight of a curing agent and the aqueous silver-containing solution of the present invention in an amount such that a concentration of silver is in a range of 5 to 100 ppm.

In accordance with yet another aspect of the present invention, there is provided an antibacterial resin-coated steel plate having a dry film thickness of not more than 5 □ which is formed by coating of the aqueous antibacterial resin composition of the present invention.

### BEST MODE FOR CARRYING OUT THE INVENTION

Hereinafter, the present invention will be described in more detail.

The present invention enables improvement in antibacterial activity, corrosion resistance, conductivity and adhesion of a steel plate by coating a steel plate with an aqueous antibacterial resin composition which is prepared via combination of a silver-containing solution having a controlled content of impurities below a predetermined level with a certain resin.

Generally, silver (Ag), copper (Cu), zinc (Zn) and the like are metals known to exhibit antibacterial activity. Inter alia, Ag exerts excellent sterilizing activity. Meanwhile, as a method for split of silver into nano-sized particles, there may be generally employed a method for split of silver from silver salts utilizing an adsorbed surfactant (hereinafter, referred to as “adsorption method” and a method of preparing a polymer-silver nanocomposite using silver salts and other polymers (hereinafter, referred to as “composite method”).

In the above-mentioned adsorption method, fine silver particles are prepared from the silver salts utilizing the adsorbed surfactant. That is, according to the adsorption method, when silver ions in the silver salts are reduced into silver particles, the size and distribution of metal particles are controlled by the surfactant. Due to addition of an aqueous surfactant solution upon preparation of silver particles using the silver salts and unique properties of the adsorbed surfactant, the surfactant is adsorbed on surfaces of the thus-formed silver particle nuclei, in the solution where metal particles are formed, which thereby results in prevention of fusion between silver particle nuclei.

Consequently, binding of reduced metal particles to surfaces of other metal particle nuclei is delayed or blocked and therefore metal particles having uniform size distribution and fine size are prepared. In the adsorption method, silver salts such as AgNO₃ and the surfactant are used, and metal ion reducing agents such as hydrazine, lithium aluminum borohydride, sodium borohydride and ethylene oxide are also added. Preparation of Fine Silver Particles Via the Use of Such an Adsorption Method is Fully described in Korean Patent Registration No. 375525.

Alternatively, according to split of silver particles by the composite method, silver metals are reduced into fine particles by formation of a polymer-silver nanocomposite using silver salts and polymer materials. Specifically, in the composite method, silver particles are prepared by mixing a silver salt or silver oxide salt, isopropyl alcohol, ethanol and/or ethylene glycol solvents and water, and a polymeric stabilizer, purifying the resulting mixture, forming precipitates and removing the solvents.

The silver salt that can be used in the present invention may be at least one selected from the group consisting of silver nitrate (AgNO₃), silver perchlorate (AgClO₄), silver sulfate (AgSO₄) and silver acetate (CH₃COOAg). The polymeric stabilizer that can be used in the present invention may be at least one selected from the group consisting of polyethylene, polyacrylonitrile, polymethylmethacrylate, polyurethane, polyacrylamide, polyethylene glycol and polyoxyethylene stearate. In order to meet both physical properties of dispersibility and stability, it is preferred to use the polymeric stabilizer having a molecular weight of 35,000 to 120,000.

Furthermore, it is also possible to obtain an emulsion using a surfactant, for example polyoxyethylene sorbitan monooleate, upon mixing of the silver salt or the like. Preparation of nano silver composite via the use of the composite method is specifically described in Korean Patent Application No. 2002-20593.

Silver salts, surfactants, metal ion reducing agents and the like are used in the adsorption method, while silver salts, polymeric stabilizers, surfactants, solvents and the like are used in the composite method. Anionic parts of the silver salts used, i.e., NO₃⁻ for silver nitrate (AgNO₃), Cl⁻ for silver chloride (AgCl), SO₄²⁻ for silver sulfate (AgSO₄) and CH₃COO⁻ for silver acetate (CH₃COOAg), polymeric stabilizers as an impurities, surfactants, metal ion reducing agents, water-insoluble solvents and the like remain in the resulting product, i.e., silver-containing solution, after formation of fine silver particles.

However, when the resulting silver-containing solution in admixture with a resin composition is applied to a steel plate, the remaining other components with exception of silver (Ag) and water may serve as impurities having adverse effects on physical properties of the steel plate coated with a thin film of the resin, such as corrosion resistance, conductivity and adhesion thereof, and therefore should be removed.

Therefore, in order to prevent deterioration of physical properties of the steel plate such as corrosion resistance, conductivity and adhesion, upon application of a silver-containing solution as a resin thin film to the steel plate, the present invention provides the silver-containing solution in which the contents of impurities such as anionic parts of silver salts, surfactants, metal ion reducing agents and polymeric stabilizers are controlled.

The silver-containing solution of the present invention contains silver (Ag) particles having a particle diameter of 1.0 to 20 nm at a concentration of 200 to 100,000 ppm, and preferably 1,000 to 100,000 ppm. If the diameter of silver (Ag) particles in the silver-containing solution is less than 1.0 mm, it is undesirable due to increased costs by preparation of nano silver. Conversely, if the diameter of silver (Ag) particles exceeds 20 nm, it is difficult to achieve sufficient antibacterial effects due to non-uniform distribution of nano silver upon addition thereof to a resin, and poor adhesion and corrosion resistance are obtained due to low miscibility between silver particles and resin, thereby leading to increases in costs.

In addition, if the concentration of silver in the silver-containing solution is less than 200 ppm, large amounts of the silver-containing solution should be used in order to maintain the concentration of silver relative to the resin at a predetermined level upon preparation of the resin composition and therefore physical properties of the resin are deteriorated, leading to decreased corrosion resistance and conductivity. If the concentration of silver exceeds 100,000 ppm, large amounts of the stabilizer are required in order to ensure that nano-sized silver particles are stable without causing any adverse reaction or aggregation, and as a result, corrosion resistance is undesirably decreased upon application of the silver-containing solution to the steel plate.

The acidity (pH) of the silver-containing solution should be maintained in a range of 6.0 to 8.5. If the pH of the solution is outside the above-mentioned range of 6.0 to 8.5, this leads to changes in physical properties of the waterborne resin with occurrence of aggregation thereof, thus resulting in poor spreadability of the resin and therefore low adhesion properties.

In addition, based on the weight of the silver-containing solution, the contents of impurities in the silver-containing solution should be in specified ranges, as discussed hereinbefore, i.e., 0.5 to 1.5% by weight for the stabilizer and 1.0% by weight or less for anionic parts in the silver salt, and the sum of the stabilizer and anionic parts of the silver salt as impurities should be not more than 2.0% by weight.

If the content of the stabilizer impurity in the silver-containing solution is less than 0.5% by weight, it is difficult to exert sufficient antibacterial effects due to aggregation of nano silver particles. Conversely, if the content of the stabilizer exceeds 1.5% by weight, bondability to the resin is poor and corrosion resistance is degraded. Further, if an amount of the anionic parts in the silver salt exceeds 1.0% by weight, or the sum of contents of the stabilizer and anionic parts of the silver salt as impurities exceeds 2.0% by weight, adhesion between the aqueous resin composition and steel plate is decreased and chain linkages of the resin are broken down, thereby resulting in lowering of anti-corrosion effects and conductivity of the thin film of the resin.

As used herein, the term “stabilizer as an impurity” refers to any material that may be present in the silver-containing solution, excluding the silver, water and anionic parts of the silver salt in the silver-containing solution. That is, the term “stabilizer” is understood to mean any material in the silver-containing solution, which is used in the preparation of fine silver particles via the adsorption method or composite method and includes, but is not limited to, for example, surfactants, metal ion reducing agents, polymeric stabilizers and non-aqueous solvents, with exception of silver (Ag), water and anionic parts of the silver salt in the silver-containing solution.

Impurities including the stabilizer and anionic parts of the silver salt, which are present in the silver-containing solution, may be removed by a method utilizing an ion exchange resin or a variety of any other methods conventionally known in the art.

The silver-containing solution and antibacterial resin composition of the present invention are water-based as discussed hereinbefore. Therefore, removal of water following application thereof to the steel plate does not require any separate process and can thus be effected even with application of a high temperature only.

The aqueous antibacterial resin composition of the present invention may be comprised of 100 parts by weight of at least one resin selected from the group consisting of acrylic, urethane, epoxy and ester resins, 0.05 to 5 parts by weight of a curing agent and the silver-containing solution of the present invention in an amount such that a concentration of silver is in a range of 5 to 100 ppm.

The composition of the present invention includes the silver-containing solution in an amount such that the content of silver (Ag) is in a range of 5 to 100 ppm and preferably 20 to 100 ppm. If the content of silver is less than 5 ppm, it is difficult to exhibit sufficient antibacterial effects. Conversely, the silver content exceeding 100 ppm results in decreased adhesion and increased costs due to poor adhesiveness between silver components and resin composition.

As the resin for use in the antibacterial resin composition, at least one resin selected from the group consisting of acrylic, urethane, epoxy and ester resins may be employed alone or in any combination thereof. The above-mentioned resin materials are known as anti-fingerprint resins.

Additionally, the curing agent is added in an amount of 0.05 to 5 parts by weight per 100 parts by weight of the resin. If the content of the curing agent is less than 0.05 parts by weight, it is undesirable in that a paint film is not easily hardened. Conversely, if the content of the curing agent exceeds 5 parts by weight, addition of excess amounts of the curing agent results in brittleness of the paint film and poor processability, and it is also non-economical because the curing agent is expensive. As the curing agent, aziridine or melamine may be used.

In order to improve corrosion resistance and adhesion properties of the paint film upon application of the antibacterial resin composition to the steel plate, colloidal silica may be additionally added to the composition. Colloidal silica imparts irregularities on the surface of the coated film, and improves adhesion and corrosion resistance of the film via anchoring effects. Preferably, 0.5 to 100 parts by weight of colloidal silica are added based on 100 parts by weight of the composition with exception of the silver-containing solution from the aqueous antibacterial resin composition of the present invention. The silica content of less than 0.5 parts by weight does not exhibit desired corrosion resistance and adhesion enhancing effects. In addition, the silica content of greater than 100 parts by weight leads to an increase in a friction coefficient upon processing and brittleness of the paint film, thereby resulting in deterioration of processability. Therefore, the colloidal silica should be added in the above-specified range of 0.5 to 100 parts by weight.

Further, in order to improve conductivity, a metal component may be optionally added to the resin composition, if necessary. The metal component is an optional component which is used to additionally enhance conductivity and therefore may be not added, but, if desired, it may be added in an amount of up to 3 parts by weight, based on 100 parts by weight of the composition with exception of the silver-containing solution from the aqueous antibacterial resin composition of the present invention. If the content of the metal component is higher than 3 parts by weight, surface cracks of the coating film occur due to the presence of the metal particles when the composition is applied as a resin layer to the steel plate, which consequently results in deterioration of corrosion resistance and surface appearance. The metal component that can be used in the composition of the present invention may be at least one selected from the group consisting of titanium (Ti), zirconium (Zr) and manganese (Mn).

In addition, if necessary, wax may be additionally added to the aqueous antibacterial resin composition, in order to improve lubricability thereof. Wax is also an optional component which is used to additionally enhance lubricability of the composition and therefore may be not added, but, if desired, it may be added in an amount of up to 7 parts by weight, based on 100 parts by weight of the composition with exception of the silver-containing solution from the aqueous antibacterial resin composition of the present invention. If the content of added wax exceeds 7 parts by weight, adhesion of the resin layer being formed is undesirably decreased.

The aqueous antibacterial resin composition is a water-based resin composition and the content of solids therein is preferably in a range of 5 to 30% by weight. When the content of solids is less than 5% by weight, it is difficult to control a thickness of the paint film and there is also a risk of a decrease in corrosion resistance. Conversely, when the content of solids is higher than 30% by weight, there are also difficulties associated with the control of a film thickness and poor workability.

In addition, the antibacterial resin composition, if necessary, may further include other additives such as a dispersant, a leveling agent and a thickener, and coloring pigments, which are conventionally added in preparation of a composition for coating steel plates and are well-known in the art.

The antibacterial resin composition according to the present invention is coated as a thin film layer of the resin on the steel plate and thereby imparts antibacterial activity, corrosion resistance, adhesion and conductivity thereto.

The steel plate that can be used in the present invention may be common one and preferably a zinc-galvanized steel plate may be employed. After galvanization of the steel plate, a prime-coated steel plate may also be employed. Prime coating is a common process in the related art and may be carried out using, for example at least one resin selected from the group consisting of acrylic, urethane, epoxy and ester resins, as discussed hereinbefore.

The antibacterial resin composition of the present invention is coated on one side of the steel plate, as a thin film having a dry film thickness of not more than 5 □ and preferably 1 to 2 □, thereby forming a resin coating layer. The resin coating layer is preferably formed as thin a thickness as possible. However, taking into consideration roughness of the steel plate and the like, the coating layer may be formed to have a dry film thickness of about 1 □. Further, a thinner film may also be applied to the substrate by stricter control of the silver content. However, if the dry resin film thickness exceeds 5 □, it is non-economical and there may also occur problems associated with decreased conductivity and adhesion, which are typically suffered by conventional thick film-type steel plates.

Furthermore, the resin composition of the present invention exhibits excellent corrosion resistance, and therefore is preferred with superior corrosion resistance particularly when it is desired to form a resin layer having a dry film thickness of not more than 5 □ which typically suffers from the problems associated with poor anticorrosiveness.

Nano silver exhibits antibacterial/sterilizing action by silver cations (Ag⁺) having antibacterial activity. More specifically, such antibacterial/sterilizing action of nano silver is effected by the following action mechanisms:

Silver cations (Ag⁺) directly and strongly bind to —SH, —COOH and —OH groups, which are present on bacteria, thereby destroying cell membranes or disturbing cellular functions, or silver in the nano state catalyzes conversion of oxygen into active oxygen species having sterilizing action, thereby exerting antibacterial functions via sterilizing mechanisms of active oxygen species.

The steel plate, on which a coating film was formed by application of the antibacterial resin composition comprising the silver-containing solution of the present invention, is a anti-fingerprint steel plate, exhibits an antibacterial rate of more than 99.9% and has corrosion resistance, conductivity and adhesion comparable to those of conventional anti-fingerprint steel plates.

As described hereinabove, the present invention imparts superior antibacterial activity, corrosion resistance, conductivity and adhesion to a steel plate of interest via adjustment of a size of silver particles, a silver content, acidity (pH) and amounts of impurities in the silver-containing solution, and amounts of silver and curing agent in the antibacterial resin composition to within the above-specified ranges.

### MODE FOR THE INVENTION

Now, the present invention will be described in more detail with reference to the following example. This example is provided only for illustrating the present invention and should not be construed as limiting the scope and spirit of the present invention.

### EXAMPLE

Aqueous silver-containing solutions 1 through 27 were respectively prepared by adjusting a concentration of an anionic part of a silver salt, a concentration of a stabilizer as an impurity, a total concentration of the anionic part of the silver salt and stabilizer as an impurity, acidity (pH) and a size of silver particles to corresponding ranges as set forth in Table 1 below and mixing the respective components. The content of silver in the silver-containing solutions was set to 10,000 ppm. Upon preparation of the silver-containing solutions in this example, silver nitrate was used as the silver salt and a mixture of polyethylene having a molecular weight of 50,000 and polymethylmethacrylate having a molecular weight of 100,000 in a 9:1 weight ratio was used as the stabilizer.

After preparation of silver-containing solutions 1 through 27 as listed in Table 1, aqueous antibacterial resin compositions 1 through 27 were prepared by adding each silver-containing solution to the corresponding resin compositions such that the silver contents were in the ranges given in Table 1, followed by addition of 1 part by weight of aziridine as the curing agent per 100 parts by weight of a resin, and 20 parts by weight of colloidal silica per 100 parts by weight of the composition with exception of the silver-containing solution. Contents of solids in the aqueous antibacterial resin compositions 1 through 27 were 15% by weight, and an epoxy resin was used as the resin.

Using a roll coating method, each of the thus-prepared antibacterial resin compositions was applied to one side of the corresponding electro-galvanized (EG) steel plates having a thickness of 0.8 mm, such that coating having a dry film thickness of 1 □ is formed on EG steel plates and a plating amount per one side of the steel plate is 20 g/m². Then, the thus-coated steel plates were dried in a drying furnace at 150° C. for a sufficient period of time and were subjected to water cooling to thereby form resin films. Next, antibacterial activity, corrosion resistance, conductivity and adhesion of the respective steel plates were measured. The results thus obtained are given in Table 1 below.

1. Evaluation of Antibacterial Activity (E. coli and Staphylococci)

Escherichia coli (ATCC 25922) and Staphylococcus aureus (ATCC 6538) were incubated in a general incubator at 35° C. for 16 to 20 hours, and the cell culture was diluted 20,000-fold with sterile phosphate buffer solution (PBS). 1 mL of the diluted culture was added dropwise to the resin-treated specimen which was then stored at 25° C. for 24 hours. Thereafter, bacterial cells of the specimen were cultured again on a sterile agar medium for 48 hours and viable cell count (VC) was measured by plate culture (35° C., 2 days). Next, a sterilization rate (%) was calculated according to the following equation. In Table 1, respective symbols represent the following meanings: ⊚: 99.9% or higher; ◯: 99% or higher; Δ: 80% or higher; and □: less than 80%:

Sterilization rate (%)=[(viable cell counts in diluted solution−viable cell counts after 24-hour storage)/viable cell counts in diluted solution]×100

2. Evaluation of Corrosion Resistance

Corrosion resistance was evaluated by performing Salt Spray Test (JIS K5400) for 100 hours. In Table 1, respective symbols represent the following meanings: ⊚: no occurrence of white rust; ◯: 5% or less; and □: higher than 5%.

3. Evaluation of Conductivity

Conductivity was evaluated by measuring surface resistance of steel plates after resin treatment thereof. In Table 1, respective symbols represent the following meanings: based on resistance values observed between a distance of 1 cm, ⊚: 0.04 mΩ or less; ◯: 0.04 to 0.06 mΩ and □: higher than 0.06 mΩ.

4. Evaluation of Adhesion

Adhesion was evaluated by a taping test involving bending a test specimen to 180° attaching a cellophane tape to the bent part and detaching the tape therefrom. The results are shown in Table 1 as follows: ⊚: non-peeling of a paint film; ◯: peeling of 5% or less; and □: peeling of more than 5%.

TABLE 1

| | | | | | | | Plating quality | | | |
|---|---|---|---|---|---|---|---|---|---|---|
| | Silver-containing solution | | | | | Amount | Antibacterial | | | |
| | | Conc. of | | | Particle | of Ag | activity 1) | Corrosion | | |
| Composition | Conc. of anions in | stabilizer | A + B | Acidity | size of | added | (E. coli | resistance | Conductivity | Adhesion |
| No. | Ag salt (wt %): A | (wt %): B | (wt %) | (pH) | Ag (mm) | (ppm) | Staphylococci) | 2) | 3) | 4) |
| 1 | 0.2 | 0.6 | 0.8 | 7 | 7 | 50 | ⊚ | ⊚ | ⊚ | ⊚ |
| 2 | 0.3 | 0.5 | 0.8 | 7 | 7 | 50 | ⊚ | ⊚ | ⊚ | ⊚ |
| 3 | 0.5 | 0.3 | 0.8 | 7 | 7 | 50 | □ | □ | ⊚ | □ |
| 4 | 0.6 | 0.2 | 0.8 | 7 | 7 | 50 | □ | □ | □ | □ |
| 5 | 0.9 | 0.8 | 1.7 | 7 | 7 | 50 | ⊚ | ⊚ | ⊚ | ⊚ |
| 6 | 1.0 | 0.8 | 1.8 | 7 | 7 | 50 | ⊚ | ◯ | ◯ | ◯ |
| 7 | 1.1 | 0.8 | 1.9 | 7 | 7 | 50 | ⊚ | □ | □ | □ |
| 8 | 0.3 | 0.9 | 1.2 | 7 | 7 | 50 | ⊚ | ⊚ | ⊚ | ⊚ |
| 9 | 0.3 | 1.5 | 1.8 | 7 | 7 | 50 | ⊚ | ◯ | ◯ | ◯ |
| 10 | 0.3 | 1.6 | 1.9 | 7 | 7 | 50 | ◯ | □ | □ | □ |
| 11 | 0.7 | 1.2 | 1.9 | 7 | 7 | 50 | ⊚ | ⊚ | ⊚ | ⊚ |
| 12 | 0.8 | 1.2 | 2.0 | 7 | 7 | 50 | ◯ | ◯ | ◯ | ◯ |
| 13 | 0.8 | 1.3 | 2.1 | 7 | 7 | 50 | ◯ | □ | □ | □ |
| 14 | 0.5 | 1.0 | 1.5 | 5.5 | 7 | 50 | Δ | □ | ◯ | □ |
| 15 | 0.5 | 1.0 | 1.5 | 6 | 7 | 50 | ◯ | ◯ | □ | ◯ |
| 16 | 0.5 | 1.0 | 1.5 | 8 | 7 | 50 | ⊚ | ⊚ | ⊚ | ⊚ |
| 17 | 0.5 | 1.0 | 1.5 | 8.5 | 7 | 50 | ◯ | ◯ | ◯ | ◯ |
| 18 | 0.5 | 1.0 | 1.5 | 8.7 | 7 | 50 | Δ | □ | □ | □ |
| 19 | 0.5 | 1.0 | 1.5 | 7 | 5 | 50 | ⊚ | ⊚ | ⊚ | ⊚ |
| 20 | 0.5 | 1.0 | 1.5 | 7 | 15 | 50 | ⊚ | ⊚ | ⊚ | ⊚ |
| 21 | 0.5 | 1.0 | 1.5 | 7 | 20 | 50 | ◯ | ⊚ | ⊚ | ◯ |
| 22 | 0.5 | 1.0 | 1.5 | 7 | 22 | 50 | Δ | □ | ◯ | □ |
| 23 | 0.5 | 1.0 | 1.5 | 7 | 7 | 4 | □ | ⊚ | ◯ | ⊚ |
| 24 | 0.5 | 1.0 | 1.5 | 7 | 7 | 5 | ◯ | ⊚ | ⊚ | ⊚ |
| 25 | 0.5 | 1.0 | 1.5 | 7 | 7 | 80 | ⊚ | ⊚ | ⊚ | ⊚ |
| 26 | 0.5 | 1.0 | 1.5 | 7 | 7 | 100 | ⊚ | ◯ | ◯ | ◯ |
| 27 | 0.5 | 1.0 | 1.5 | 7 | 7 | 102 | ⊚ | □ | ◯ | □ |

As can be seen from Table 1, when silver-containing solutions and antibacterial resin compositions, which meet limitations specified by the present invention, were applied, the thus-coated steel plates exhibited excellent antibacterial activity, corrosion resistance, conductivity and adhesion. On the other hand, it was revealed that antibacterial resin compositions 3, 4, 7, 10, 13, 14, 18, 22, 23 and 27, which are outside the specified limitations of the present invention, exhibited no simultaneous satisfaction of antibacterial activity, corrosion resistance, conductivity and adhesion properties.

### INDUSTRIAL APPLICABILITY

Steel plates, which are coated with an antibacterial resin composition comprising an aqueous silver-containing solution of the present invention, exhibit superior antibacterial properties, corrosion resistance, conductivity and adhesion.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An aqueous silver-containing solution comprising nano-sized silver particles wherein silver (Ag) particles having a particle diameter of 1.0 to 20 nm have a concentration of 200 to 100,000 ppm, the pH of the solution is maintained in a range of 6 to 8.5, and based on the weight of the silver-containing solution, a content of a stabilizer as an impurity is in a range of 0.5 to 1.5% by weight, a content of an anionic part of a silver salt as another impurity is 1.0% by weight or less and the sum of the stabilizer and anionic part of the silver salt is 2.0% by weight or less.

2. An aqueous antibacterial resin composition comprising 100 parts by weight of at least one resin selected from the group consisting of acrylic, urethane, epoxy and ester resins, 0.05 to 5 parts by weight of a curing agent and a silver-containing solution of claim 1 in an amount such that a concentration of silver is in a range of 5 to 100 ppm.

3. The composition according to claim 2, further comprising 0.5 to 100 parts by weight of colloidal silica, based on 100 parts by weight of the resin composition with exception of the silver-containing solution.

4. The composition according to claim 2, further comprising at least one metallic element selected from the group consisting of titanium (Ti), zirconium (Zr) and manganese (Mn), in an amount of up to 3 parts by weight, based on 100 parts by weight of the resin composition with exception of the silver-containing solution.

5. The composition according to claim 2, further comprising 7 parts by weight or less of wax, based on 100 parts by weight of the resin composition with exception of the silver-containing solution.

6. The composition according to claim 2, wherein a solid content of the resin composition is in a range of 5 to 30% by weight.

7. The composition according to claim 2, wherein the curing agent is aziridine or melamine.

8. A steel plate having a dry film thickness of not more than 5 μm formed by coating with an aqueous antibacterial resin composition of claim 2.

9. The steel plate according to claim 8, wherein the steel plate is a zinc-galvanized steel plate or a zinc-galvanized and prime-coated steel plate.

10. A steel plate having a dry film thickness of not more than 5 μm formed by coating with an aqueous antibacterial resin composition of claim 3.

11. A steel plate having a dry film thickness of not more than 5 μm formed by coating with an aqueous antibacterial resin composition of claim 4.

12. A steel plate having a dry film thickness of not more than 5 μm formed by coating with an aqueous antibacterial resin composition of claim 5.

13. A steel plate having a dry film thickness of not more than 5 μm formed by coating with an aqueous antibacterial resin composition of claim 6.

14. A steel plate having a dry film thickness of not more than 5 μm formed by coating with an aqueous antibacterial resin composition of claim 7.
